(19) United States
(12) Patent Application Publication
(10) Pub. No.: US 2009/0299261 A1
(43) Pub. Date: Dec. 3, 2009

(54) Expandable Catheters and Methods Relating Thereto

(76) Inventor: Istvan Bognar, Greenville, SC (US)

Correspondence Address: DORITY & MANNING, P.A., POST OFFICE BOX 1449, GREENVILLE, SC, 29602-1449, US

(21) Appl. No.: 12/477,610

(22) Filed: Jun. 3, 2009

Related U.S. Application Data
(60) Provisional application No. 61/058,353, filed on Jun. 3, 2008.

(51) Int. Cl. A61M 25/10

(52) U.S. Cl. 604/6.16; 604/103.03; 604/99.01; 604/540

(57) ABSTRACT

In accordance with certain embodiments of the present disclosure, a catheter is provided. The catheter includes a body having a proximal portion and a distal portion. The body comprises a first element sealably interwoven with a second element to define a longitudinally extending lumen configured to pass fluid therethrough. The first element is wound in a clockwise direction and the second element is wound in a counterclockwise direction. The body further comprises a mechanism configured to cause the distal portion of the body to move toward the proximal portion causing the diameter of the lumen to increase.

### CROSS REFERENCE TO RELATED APPLICATION

The present application is based on and claims priority to U.S. Provisional Application Ser. No. 61/058,353 having a filing date of Jun. 3, 2008, which is incorporated by reference herein.

### BACKGROUND

Catheters are well-known medical devices that can be used to facilitate various medical procedures. A catheter can be inserted into the body of a patient and thereby allow drainage or removal of fluids or other material. In addition, catheters can be utilized for delivery of fluids and can also be used for access inside a patent with surgical instruments.

For instance, a typical internal urinary catheter is known as a Foley catheter. Such a catheter includes a hollow tube having a tip at one end which is inserted into the body. The tip has one or more openings that communicate with the interior of the hollow tube. The other end of the tube is disposed externally of the body and is connected to a waste receptacle by way of a flexible discharge conduit. An annular inflatable portion, or balloon, is spaced inwardly from the tip. The balloon is deflated during placement of the catheter into the body and is thereafter inflated after the tip is properly positioned within the body in the urinary bladder of the patient.

A problem arises if unintended force is applied to the catheter. With the balloon fully inflated, the pulling or dislodging of the catheter from its location in the urinary bladder and through the urethra (urinary canal) can cause severe lacerations, bleeding, and the possibility of damage to the external urinary sphincter with permanent urinary incontinence. Any displacement of the balloon also will cause severe pain to the patient. Even if the inflated balloon is not pulled into or through the urinary canal, the mere rotation of the hollow tube within the urinary canal will cause severe pain to the patient.

Another type of catheter, a venous access catheter, provides venous access to the central circulatory system. Venous access catheters include central venous catheters, dialysis catheters and peripherally inserted central catheters, also known as PICC lines. Such catheters can be used for the delivery of intravenous fluids, medications such as chemotherapy drugs and antibiotics, and blood products. Venous access catheters can also be used as access mechanisms for blood sampling and the administration of contrast agents during diagnostic procedures.

In navigating vessels to access a target site of a patient, the smallest catheter profile (i.e. the smallest outer diameter catheter body) is desirable. Such a minimal profile facilitates insertion through smaller vessels as it reduces the likelihood of the catheter engaging the wall of the vessel and reduces trauma to the vessel by minimizing frictional contact with the vessel wall. However, the desire for smaller diameter catheters must be balanced against the need for providing sufficient sized lumens to enable proper blood flow. If the lumens are too small, sufficient blood flow may not be able to be maintained and the blood can be damaged during transport. Unfortunately, presently available catheters fail to adequately balance the need for a minimal profile with the need for a sufficiently sized lumen.

The aforementioned problems and concerns limit the effectiveness of presently available catheters. Thus, a need exists for an expandable catheter that can utilized in a variety of applications in an efficient and cost effective manner. Methods relating to such a catheter would also be desirable.

### SUMMARY

In accordance with one embodiment of the present disclosure, a catheter is provided. The catheter includes a body having a proximal portion and a distal portion. The body comprises a first element sealably interwoven with a second element to define a longitudinally extending lumen configured to pass fluid therethrough. The first element is wound in a clockwise direction and the second element is wound in a counterclockwise direction. The body further comprises a mechanism configured to cause the distal portion of the body to move toward the proximal portion causing the diameter of the lumen to increase.

In another embodiment of the present disclosure, a catheter is provided. The catheter includes a body having a proximal portion and a distal portion. The body comprises a first element sealably interwoven with a second element to define a longitudinally extending lumen configured to pass fluid therethrough. The first element is wound in a clockwise direction and the second element is wound in a counterclockwise direction. The body comprises a mechanism configured to cause the distal portion of the body to move toward the proximal portion causing the diameter of the lumen to increase. The body further comprises a second longitudinally extending lumen configured to pass fluid therethrough, the second longitudinally extending lumen being positioned within the first longitudinally extending lumen.

In yet another embodiment of the present disclosure, a method for using a catheter is provided. The method includes inserting a catheter into a patient and actuating the mechanism to cause the distal portion of the body to move toward the proximal portion causing the diameter of the lumen to increase.

Other features and aspects of the present disclosure are discussed in greater detail below.

### BRIEF DESCRIPTION OF THE DRAWINGS

A full and enabling disclosure, including the best mode thereof, directed to one of ordinary skill in the art, is set forth more particularly in the remainder of the specification, which makes reference to the appended figures in which:

FIG. 1 illustrates a view of a venous access catheter in accordance with certain embodiments of the present disclosure;

FIG. 2 illustrates a view of a venous access catheter in an expanded position in accordance with certain embodiments of the present disclosure;

FIG. 3 illustrates a view of a urinary catheter in accordance with certain embodiments of the present disclosure;

FIG. 4 illustrates a view of a urinary catheter in an expanded position in accordance with certain embodiments of the present disclosure; and

FIG. 5 illustrates a portion of a body of a catheter in accordance with certain embodiments of the present disclosure.

Repeat use of reference characters in the present specification and drawings is intended to represent same or analogous features or elements of the disclosure.

### DETAILED DESCRIPTION

Reference now will be made in detail to various embodiments of the disclosure, one or more examples of which are set forth below. Each example is provided by way of explanation of the disclosure, not limitation of the disclosure. In fact, it will be apparent to those skilled in the art that various modifications and variations can be made in the present disclosure without departing from the scope or spirit of the disclosure. For instance, features illustrated or described as part of one embodiment, can be used on another embodiment to yield a still further embodiment. Thus, it is intended that the present disclosure covers such modifications and variations as come within the scope of the appended claims and their equivalents.

The present disclosure is generally directed to an expandable catheter. The catheter can be radially expanded with a mechanism that causes a distal portion of the catheter to move toward a proximal portion. In this manner, the catheters described herein have a smaller profile when being inserted in a patient and can be enlarged after insertion. In particular, the catheters of the present disclosure can be utilized as venous access catheters or urinary catheters. However, while the catheters of the present disclosure will be described in connection with particular applications, embodiments of the catheters described herein can be applied to, or used in connection with, numerous other applications. Moreover, the mechanism of the present disclosure can be used in combination with existing medical devices known in the art.

Referring to FIG. 1, a venous access catheter 10 in accordance with the present disclosure is illustrated in a non-expanded configuration. The catheter 10 includes a body 20 having a proximal portion 22 and distal portion 24. The body 20 includes a first element that is sealably interwoven with a second element to define a longitudinally extending lumen 14.

Turning to FIG. 5, first element 30 and second element 32 can each be generally helical in shape. In certain embodiments, first element 30 and second element 32 can be formed from one or more fiber materials. For instance, suitable materials can include polyamides, polyolefins, polyesters, viscose polymers, acetate, cotton, wool, silk, and combinations thereof. First element 30 and second element 32 are interwoven or braided such that the rotational direction of each of the elements 30, 32 can be opposite one another. In this manner, when the elements 30, 32 are axially compressed along the longitudinal axis defined by the elements 30, 32, body 20 radially expands and the diameter of lumen 14 expands.

In certain embodiments, one of the elements can be wound in a clockwise direction, while the other element is wound in a counterclockwise direction. In such an arrangement, when axial compressive force is applied to elements 30, 32 along their longitudinal axis, the elements 30, 32 transfer at least a portion of the applied compressive force against each other, and the resultant force is directed radially outwardly. In addition, the elements 30, 32 are interwoven such that the lumen 14 defined by the elements 30, 32 can accommodate fluid without leaking. Importantly, body 20 is configured so that fluids can pass therethrough without the need for elements 30, 32 to be positioned within another lumen. Rather, elements 30, 32 serve to define the lumen within which fluids can flow through.

In this regard, the spaces or interstices between elements 30, 32 can be filled with any suitable material as would be known in the art that is flexible enough to allow movement of the elements 30, 32 yet sufficiently thick enough and durable enough to prevent fluid from passing therethrough. Suitable materials for filling such spaces can include polymeric materials as would be known in the art, such as tetrafluroethylene polymers such as Teflon(polytetrafluoroethylene), insoluble synthetic resins including polyolefins such as polyethylene, polypropylene and the like, polydiolefins such as polymers of butadiene and isoprene, polystyrene, polyesters, polyamides, aqueous dispersions thereof, and combinations thereof. Such polymeric materials can be applied as a polymer coating on the body 20.

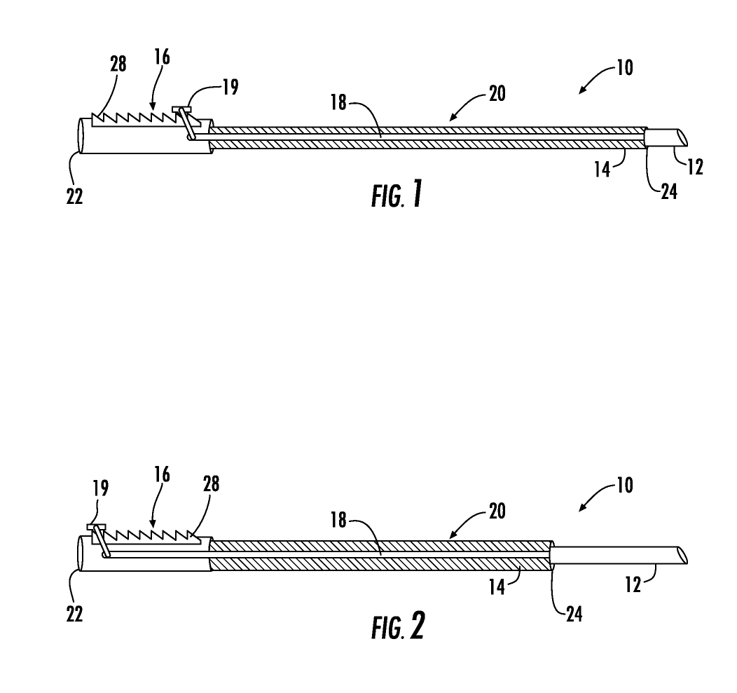

Referring again to FIG. 1, the body 20 includes a mechanism 16 that can actuate expansion of the body 20. The mechanism 16 can be any suitable mechanism that can move a proximal portion 22 of the body 20 towards a distal portion 24 of the body. The ratio of expansion of body 20 from an unexpanded configuration (FIG. 1) to an expanded configuration (FIG. 2) can depend on a number of factors, such as the length of body 20, the angle of elements 30, 32, the material chosen for elements 30, 32, and the magnitude of force applied from mechanism 16. As depicted in FIGS. 1 and 2, mechanism 16 is a latching mechanism in which arm 18 is joined to latch 19. In this manner, the degree of expansion can be controlled based on the position of latch 19. However, it should be understood that any suitable mechanism 16 as would be known in the art can be utilized to actuate movement of the proximal portion 22 of the body 20 towards a distal portion 24 of the body.

As indicated above, FIGS. 1 and 2 depict a venous access catheter in unexpanded and expanded configurations, respectively. As would be known and appreciated in the art, such catheters can include multiple lumens. For instance, as shown in FIGS. 1 and 2, a second longitudinally extending lumen 12 is positioned within the lumen 14 defined by elements 30, 32. Depending on the specific application, proximal end 22 and distal end 24 can be joined to appropriate attachments as would be known in the art. For example, a dialysis catheter embodiment could contemplate proximal end portion 22 joined to a hub or port having multiple openings (not shown) where the lumens formed within body 20 are connected, i.e. transition, to respective inflow and outflow tubes to enable return and withdrawal of blood for dialysis. In this manner, the catheters of the present disclosure can advantageously be inserted while in an unexpanded orientation, yet expand to provide a lumen that is large enough to accommodate the desired amount of fluid. However, any suitable configuration is contemplated by the present disclosure.

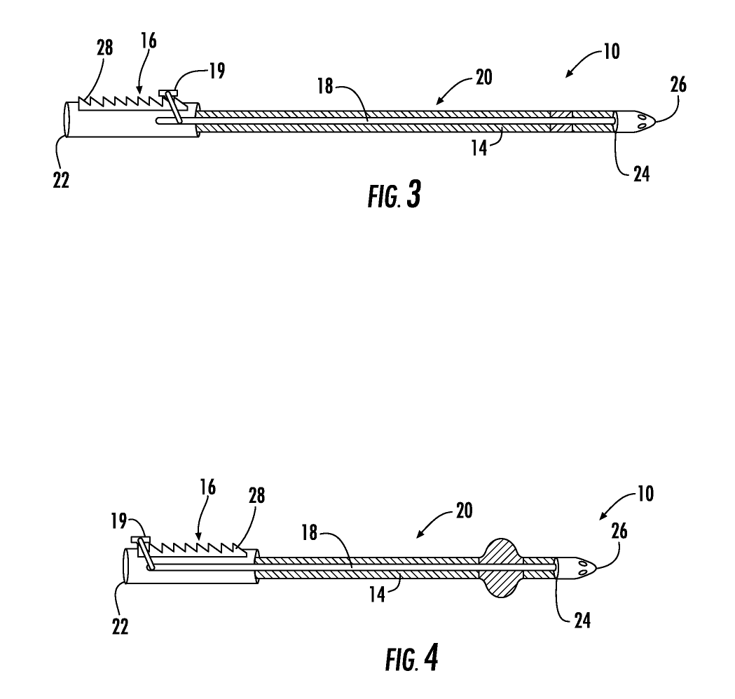

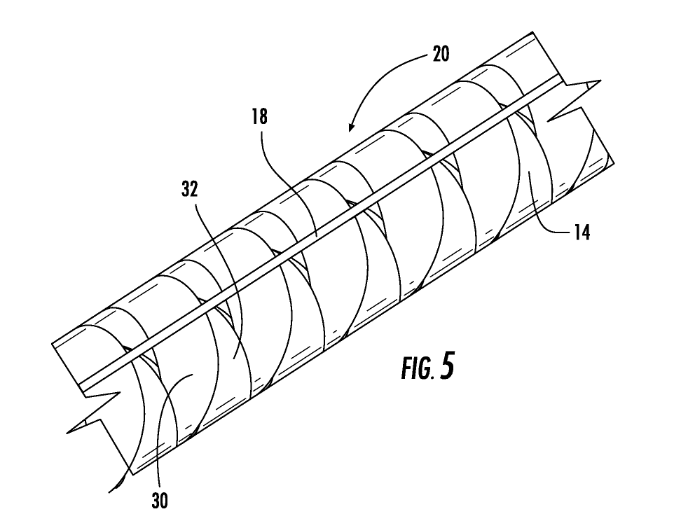

Referring to FIGS. 3 and 4, a urinary catheter 10 in accordance with the present disclosure is illustrated. The urinary catheter 10 is shown in an unexpanded configuration (FIG. 3) and an expanded configuration (FIG. 4). The catheter 10 includes a body 20 having a proximal portion 22 and distal portion 24. The body 20 includes a first element that is sealably interwoven with a second element to define a longitudinally extending lumen 14. As previously described, the body 20 includes a mechanism 16 that can actuate expansion of the body 20 by moving proximal portion 22 of the body 20 towards distal portion 24 of the body. Distal portion 24 of body 20 can be joined to tip 26 which is configured to receive urine. Proximal end 22 of body 20 can be joined to a waste receptacle (not shown) by way of a flexible discharge conduit.

As illustrated in FIG. 4, a catheter in accordance with the present disclosure can be configured so that only a portion of body 20 expands when actuated by mechanism 16. This can be accomplished by joining a segment formed with first element and second element between two segments of conventional catheter tubing. Alternatively, the body can comprise first element and second element for substantially the entire length of lumen and the angle of elements and/or the material chosen for elements in a certain segment can be configured to expand to a greater degree than the surrounding portions of the lumen. Regardless of the mechanism employed, the catheters of the present disclosure can advantageously expand to hold the catheter in place after insertion.

In this regard, a problem with existing catheters is that unintended force being applied to the catheter can cause complications for the patient including severe lacerations, bleeding, and the possibility of damage to the external urinary sphincter with permanent urinary incontinence. The catheters of the present disclosure can be configured so as to trigger mechanism 16 to return to an unactuated state and thereby return the catheter to an unexpanded configuration if there is any sudden pulling force applied to catheter. Referring to FIGS. 3 and 4, mechanism 16 is a latching mechanism in which arm 18 is joined to latch 19. The teeth 28 which engage latch 19 can be configured to provide a minimum amount of resistance so that if the pressure on catheter rises above a predetermined minimum amount, the catheter will return to an unexpanded configuration.

The catheters described herein can also optionally include a treatment on the exterior and/or the interior surfaces. The surface treatments can include, for example, hydrophilic coatings to increase lubricity and facilitate insertion of the catheter in a patient, drug coatings such as heparin or containing platelet inhibitors, inert coating substances such as Sorins carbon coating, and/or active coatings such as a silver ion coating, antibacterial coatings, or combinations thereof.

It should be understood that the catheters described herein can be of any suitable length and or width to properly serve the purpose for which they are designed. One of ordinary skill in the art will appreciate that the measurements of the components described herein can vary. However, in certain embodiments, the body of the catheter described herein can have a length of from about 5 cm to about 50 cm and a diameter of from about 0.1 cm to about 1 cm.

Methods of utilizing the catheters of the present disclosure will now be discussed. The methods described herein can be completed by any trained user of the catheter, such as a technician or doctor, or can even be self-administered by a patient. The catheters described herein can be inserted in a patient as would be known and understood by one in the art. Once inserted, the mechanism can be actuated to cause the distal portion of the body of the catheter to move toward the proximal portion. Depending on the particular mechanism employed, the user can actuate the mechanism to expand the catheter. For instance, as illustrated in FIGS. 2 and 4, the user can pull the latching mechanism 16, thereby pulling arm 18, and engage latch 10 with teeth 28 at the desired level of expansion. In order to return the catheter to an unexpanded configuration, the user can return the mechanism to the previous position.

In the interests of brevity and conciseness, any ranges of values set forth in this specification are to be construed as written description support for claims reciting any sub-ranges having endpoints which are whole number values within the specified range in question. By way of a hypothetical illustrative example, a disclosure in this specification of a range of 1-5 shall be considered to support claims to any of the following sub-ranges: 1-4; 1-3; 1-2; 2-5; 2-4; 2-3; 3-5; 3-4; and 4-5.

These and other modifications and variations to the present disclosure can be practiced by those of ordinary skill in the art, without departing from the spirit and scope of the present disclosure, which is more particularly set forth in the appended claims. In addition, it should be understood that aspects of the various embodiments can be interchanged both in whole or in part. Furthermore, those of ordinary skill in the art will appreciate that the foregoing description is by way of example only, and is not intended to limit the disclosure.

What is claimed is:

1. A catheter comprising:
a body having a proximal portion and a distal portion, the body comprising a first element sealably interwoven with a second element to define a longitudinally extending lumen configured to pass fluid therethrough, the first element being wound in a clockwise direction and the second element being wound in a counterclockwise direction, the body further comprising a mechanism configured to cause the distal portion of the body to move toward the proximal portion causing the diameter of the lumen to increase.

2. The catheter of claim 1, further comprising a polymer coating, the polymer coating sealably filling any space between the interwoven first element and second element.

3. The catheter of claim 1, wherein the mechanism is further configured cause the distal portion of the body to move away from the proximal portion causing the diameter of the lumen to decrease.

4. The catheter of claim 1, wherein the first element and the second element are formed from fiber material.

5. The catheter of claim 1, wherein the catheter is a urinary catheter.

6. The catheter of claim 5, wherein the distal end is joined to a tip configured to receive urine.

7. The catheter of claim 1, wherein the catheter is a hemodialysis catheter.

8. The catheter of claim 7, further comprising a second longitudinally extending lumen configured to pass fluid therethrough, the second longitudinally extending lumen being positioned within the first longitudinally extending lumen.

9. The catheter of claim 1, wherein the proximal end is joined to a port.

10. The catheter of claim 9, wherein the port comprises a plurality of openings.

11. The catheter of claim 1, wherein the body further comprises tubing, the tubing being in fluid communication with the longitudinally extending lumen.

12. A catheter comprising:
a body having a proximal portion and a distal portion, the body comprising a first element sealably interwoven with a second element to define a first longitudinally extending lumen configured to pass fluid therethrough, the first element being wound in a clockwise direction and the second element being wound in a counterclockwise direction, the body comprising a mechanism configured to cause the distal portion of the body to move toward the proximal portion causing the diameter of the lumen to increase, the body further comprising a second longitudinally extending lumen configured to pass fluid therethrough, the second longitudinally extending lumen being positioned within the first longitudinally extending lumen.

13. The catheter of claim 12, further comprising a polymer coating, the polymer coating sealably filling any space between the interwoven first element and second element.

14. The catheter of claim 12, wherein the mechanism is further configured cause the distal portion of the body to move away from the proximal portion causing the diameter of the lumen to decrease.

15. The catheter of claim 12, wherein the first element and the second element are formed from fiber material.

16. The catheter of claim 1, wherein the catheter is a hemodialysis catheter.

17. The catheter of claim 1, wherein the proximal end is joined to a port that comprises a plurality of openings.

18. A method for using a catheter comprising:
inserting a catheter into a patient, the catheter comprising a body having a proximal portion and a distal portion, the body comprising a first element sealably interwoven with a second element to define a longitudinally extending lumen configured to pass fluid therethrough, the first element being wound in a clockwise direction and the second element being wound in a counterclockwise direction, the body further comprising a mechanism configured to cause the distal portion of the body to move toward the proximal portion causing the diameter of the lumen to increase;
actuating the mechanism to cause the distal portion of the body to move toward the proximal portion causing the diameter of the lumen to increase.

19. The method of claim 18, further comprising actuating the mechanism to cause the distal portion of the body to move away from the proximal portion causing the diameter of the lumen to decrease.

20. The method of claim 18, wherein the catheter further comprises a second longitudinally extending lumen configured to pass fluid therethrough, the second longitudinally extending lumen being positioned within the first longitudinally extending lumen.
